SEQUENCE LISTING (5 sequences)

SEQ ID NO: 1 - protein, 542 aa, Homo sapiens
MGSNKSKPKDASQRRRSLEPAENVHGAGGGAFPASQTPSKPASADGHRGPSAAFAPAAAEPKLFGGFNSSDTVTSPQRAGPLAGGVTTFVALYDYESRTETDLSFKKGERLQIVNNTRKVDVREGDWWLAHSLSTGQTGYIPSNYVAPSDSIQAEEWYFGKITRRESERLLLNAENPRGTFLVRESETTKGAYCLSVSDFDNAKGLNVKHYKIRKLDSGGFYITSRTQFNSLQQLVAYYSKHADGLCHRLTTVCPTSKPQTQGLAKDAWEIPRESLRLEVKLGQGCFGEVWMGTWNGTTRVAIKTLKPGTMSPEAFLQEAQVMKKLRHEKLVQLYAVVSEEPIYIVTEYMSKGSLLDFLKGETGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKEPEERPTFEYLQAFLEDYFTSTEPQYQPGENL

SEQ ID NO: 2 - protein, 536 aa, Homo sapiens
MGSNKSKPKDASQRRRSLEPAENVHGAGGGAFPASQTPSKPASADGHRGPSAAFAPAAAEPKLFGGFNSSDTVTSPQRAGPLAGGVTTFVALYDYESRTETDLSFKKGERLQIVNNTEGDWWLAHSLSTGQTGYIPSNYVAPSDSIQAEEWYFGKITRRESERLLLNAENPRGTFLVRESETTKGAYCLSVSDFDNAKGLNVKHYKIRKLDSGGFYITSRTQFNSLQQLVAYYSKHADGLCHRLTTVCPTSKPQTQGLAKDAWEIPRESLRLEVKLGQGCFGEVWMGTWNGTTRVAIKTLKPGTMSPEAFLQEAQVMKKLRHEKLVQLYAVVSEEPIYIVTEYMSKGSLLDFLKGETGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKEPEERPTFEYLQAFLEDYFTSTEPQYQPGENL

SEQ ID NO: 3 - protein, 541 aa, Murinae gen. sp.
MGSNKSKPKDASQRRRSLEPSENVHGAGGAFPASQTPSKPASADGHRGPSAAFVPPAAEPKLFGGFNSSDTVTSPQRAGALAGGVTTFVALYDYESRTETDLSFKKGERLQIVNNTRKVDVREGDWWLAHSLSTGQTGYIPSNYVAPSDSIQAEEWYFGKITRRESERLLLNAENPRGTFLVRESETTKGAYCLSVSDFDNAKGLNVKHYKIRKLDSGGFYITSRTQFNSLQQLVAYYSKHADGLCHRLTTVCPTSKPQTQGLAKDAWEIPRESLRLEVKLGQGCFGEVWMGTWNGTTRVAIKTLKPGTMSPEAFLQEAQVMKKLRHEKLVQLYAVVSEEPIYIVTEYMNKGSLLDFLKGETGKYLRLPQLVDMSAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKEPEERPTFEYLQAFLEDYFTSTEPQYQPGENL

SEQ ID NO: 4 - DNA, 3711 bp, Homo sapiens
catcgaggttttgagaggctaactctcccaaaaaggaccatgggtagcaacaagagcaagcccaaggatgccagccagcggcgccgcagcctggagcccgccgagaacgtgcacggcgctggcgggggcgctttccccgcctcgcagacccccagcaagccagcctcggccgacggccaccgcggccccagcgcggccttcgcccccgcggccgccgagcccaagctgttcggaggcttcaactcctcggacaccgtcacctccccgcagagggcgggcccgctggccggtggagtgaccacctttgtggccctctatgactatgagtctaggacggagacagacctgtccttcaagaaaggcgagcggctccagattgtcaacaacacagagggagactggtggctggcccactcgctcagcacaggacagacaggctacatccccagcaactacgtggcgccctccgactccatccaggctgaggagtggtattttggcaagatcaccagacgggagtcagagcggttactgctcaatgcagagaacccgagagggaccttcctcgtgcgagaaagtgagaccacgaaaggtgcctactgcctctcagtgtctgacttcgacaacgccaagggcctcaacgtgaagcactacaagatccgcaagctggacagcggcggcttctacatcacctcccgcacccagttcaacagcctgcagcagctggtggcctactactccaaacacgccgatggcctgtgccaccgcctcaccaccgtgtgccccacgtccaagccgcagactcagggcctggccaaggatgcctgggagatccctcgggagtcgctgcggctggaggtcaagctgggccagggctgctttggcgaggtgtggatggggacctggaacggtaccaccagggtggccatcaaaaccctgaagcctggcacgatgtctccagaggccttcctgcaggaggcccaggtcatgaagaagctgaggcatgagaagctggtgcagttgtatgctgtggtttcagaggagcccatttacatcgtcacggagtacatgagcaaggggagtttgctggactttctcaagggggagacaggcaagtacctgcggctgcctcagctggtggacatggctgctcagatcgcctcaggcatggcgtacgtggagcggatgaactacgtccaccgggaccttcgtgcagccaacatcctggtgggagagaacctggtgtgcaaagtggccgactttgggctggctcggctcattgaagacaatgagtacacggcgcggcaaggtgccaaattccccatcaagtggacggctccagaagctgccctctatggccgcttcaccatcaagtcggacgtgtggtccttcgggatcctgctgactgagctcaccacaaagggacgggtgccctaccctgggatggtgaaccgcgaggtgctggaccaggtggagcggggctaccggatgccctgcccgccggagtgtcccgagtccctgcacgacctcatgtgccagtgctggcggaaggagcctgaggagcggcccaccttcgagtacctgcaggccttcctggaggactacttcacgtccaccgagccccagtaccagcccggggagaacctctaggcacaggcgggcccagaccggcttctcggcttggatcctgggctgggtggcccctgtctcggggcttgccccactctgcctgcctgctgttggtcctctctctgtggggctgaattgccaggggcgaggcccttcctctttggtggcatggaaggggcttctggacctagggtggcctgagagggcggtgggtatgcgagaccagcacggtgactctgtccagctcccgctgtggccgcacgcctctccctgcactccctcctggagctctgtgggtctctggaagaggaaccaggagaagggctggggccggggctgagggtgcccttttccagcctcagcctactccgctcactgaactccttccccacttctgtgccacccccggtctatgtcgagagctggccaaagagcctttccaaagaggagcgatgggcccctggccccgcctgcctgccaccctgccccttgccatccattctggaaacacctgtaggcagaggctgccgagacagaccctctgccgctgcttccaggctgggcagcacaaggccttgcctggcctgatgatggtgggtgggtgggatgagtaccccctcaaaccctgccctccttagacctgagggacccttcgagatcatcacttccttgcccccatttcacccatggggagacagttgagagcggggatgtgacatgcccaaggccacggagcagttcagagtggaggcgggcttggaacccggtgctccctctgtcatcctcaggaaccaacaattcgtcggaggcatcatggaaagactgggacagcccaggaaacaaggggtctgaggatgcattcgagatggcagattcccactgccgctgcccgctcagcccagctgttgggaacagcatggaggcagatgtggggctgagctggggaatcagggtaaaaggtgcaggtgtggagagagaggcttcaatcggcttgtgggtgatgtttgaccttcagagccagccggctatgaaagggagcgagcccctcggctctggaggcaatcaagcagacatagaagagccaagagtccaggaggccctggtcctggcctccttccccgtactttgtcccgtggcatttcaattcctggccctgttctcctccccaagtcggcaccctttaactcatgaggagggaaaagagtgcctaagcgggggtgaaagaggacgtgttacccactgccatgcaccaggactggctgtgtaaccttgggtggcccctgctgtctctctgggctgcagagtctgccccacatgtggccatggcctctgcaactgctcagctctggtccaggccctgtggcaggacacacatggtgagcctagccctgggacatcaggagactgggctctggctctgttcggcctttgggtgtgtggtggattctccctgggcctcagtgtgcccatctgtaaaggggcagctgacagtttgtggcatcttgccaagggtccctgtgtgtgtgtatgtgtgtgcatgtgtgcgtgtctccatgtgcgtccatatttaacatgtaaaaatgtcccccccgctccgtcccccaaacatgttgtacatttcaccatggccccctcatcatagcaataacattcccactgccaggggttcttgagccagccaggccctgccagtggggaaggaggccaagcagtgcctgcctatgaaatttcaacttttcctttcatacgtctttattacccaagtcttctcccgtccattccagtcaaatctgggctcactcaccccagcgagctctcaaatccctctccaactgcctaaggccctttgtgtaaggtgtcttaatactgtcctttttttttttttaacagtgttttgtagatttcagatgactatgcagaggcctgggggacccctggctctgggccgggcctggggctccgaaattccaaggcccagacttgcggggggtgggggggtatccagaattggttgtaaatactttgcatattgtctgattaaacacaaacagacctcagaaaaaaaaaaaaaaaaaaaaaaaa

SEQ ID NO: 5 - DNA, 1626 bp, Murinae gen. sp.
atgggcagcaacaagagcaagcccaaggacgccagccagcggcgccgcagcctggagccctcggaaaacgtgcacggggcagggggcgccttcccggcctcacagacaccgagcaagcccgcctccgccgacggccaccgcgggcccagcgccgccttcgtgccgcccgcggccgagcccaagctcttcggaggcttcaactcctcggacaccgtcacctccccgcagagggcgggcgctctggcaggtggggtgaccacctttgtggccctctatgactatgagtcacggacagagactgacctgtccttcaagaaaggggagcggctgcagattgtcaataacacgaggaaggtggatgtcagagagggagactggtggctggcacactcgctgagcacgggacagaccggttacatccccagcaactatgtggcgccctccgactccatccaggctgaggagtggtactttggcaagatcactagacgggaatcagagcggctgctgctcaacgccgagaacccgagagggaccttcctcgtgagggagagtgagaccacaaaaggtgcctactgcctctctgtatccgacttcgacaatgccaagggcctaaatgtgaaacactacaagatccgcaagctggacagcggcggtttctacatcacctcccgcacccagttcaacagcctgcagcagctcgtggcttactactccaaacatgctgatggcctgtgtcaccgcctcactaccgtatgtcccacatccaagcctcagacccagggattggccaaggatgcgtgggagatcccccgggagtccctgcggctggaggtcaagctgggccagggttgcttcggagaggtgtggatggggacctggaacggcaccacgagggttgccatcaaaactctgaagccaggcaccatgtccccagaggccttcctgcaggaggcccaagtcatgaagaaactgaggcacgagaaactggtgcagctgtatgctgtggtgtcggaagaacccatttacattgtgacagagtacatgaacaaggggagtctgctggactttctcaagggggaaacgggcaaatatttgcggctaccccagctggtggacatgtctgctcagatcgcttcaggcatggcctatgtggagcggatgaactatgtgcaccgggaccttcgagccgccaatatcctagtaggggagaacctggtgtgcaaagtggccgactttgggttggcccggctcatagaagacaacgaatacacagcccggcaaggtgccaaattccccatcaagtggaccgcccctgaagctgctctgtacggcaggttcaccatcaagtcggatgtgtggtcctttgggattctgctgaccgagctcaccactaagggaagagtgccctatcctgggatggtgaaccgtgaggttctggaccaggtggagcggggctaccggatgccttgtccccccgagtgccccgagtccctgcatgaccttatgtgccagtgctggcggaaggagcccgaggagcggcccaccttcgagtacctgcaggccttcctggaagactactttacgtccactgagccacagtaccagcccggggagaacctatag

(19) United States
(12) Patent Application Publication
(10) Pub. No.: US 2004/0176313 A1
(43) Pub. Date: Sep. 9, 2004

(54) Inhibitors of Src kinase for use in alzheimer's disease

(75) Inventors: Luc Mercken, Saint Maur (FR); Nicola Zambrano, Casavatore (IT); Tommaso Russo, Napoli (IT)

Correspondence Address: ROSS J. OEHLER, AVENTIS PHARMACEUTICALS INC., ROUTE 202-206, MAIL CODE: D303A, BRIDGEWATER, NJ, 08807, US

(73) Assignee: Aventis Pharma S.A., Antony (FR)

(21) Appl. No.: 10/691,079

(22) Filed: Oct. 22, 2003

(30) Foreign Application Priority Data
Oct. 22, 2002 (EP) 02292608.3

(51) Int. Cl. C12Q 1/68; G01N 33/53; G01N 33/567; A61K 48/00

(52) U.S. Cl. 514/44; 435/6; 435/7.2

(57) ABSTRACT

The present invention relates to the identification of inhibitors of Src kinase, assays and methods useful for such identification and the use of Src inhibitors for the preparation of pharmaceuticals for the treatment of Alzheimer's disease.

The present invention relates to the identification of inhibitors of Src kinase, assays and methods useful for such identification and the use of Src inhibitors for the preparation of pharmaceuticals for the treatment of Alzheimer's disease.

Alzheimer's disease is a neurodegenerative disorder of the brain, which is accompanied at the cellular level by a massive loss of neurons in the limbic system and in the cerebral cortex. In the brain areas affected, protein deposits, so-called plaques, can be detected at the molecular level, which are an essential characteristic of Alzheimer's disease. The protein occurring most frequently in these plaques is a peptide of 40 to 42 amino acids in size, which is designated as Aβ-peptide. This peptide is a cleavage product of a larger protein of 695 to 770 amino acids, the so-called Amyloid Precursor Protein (APP).

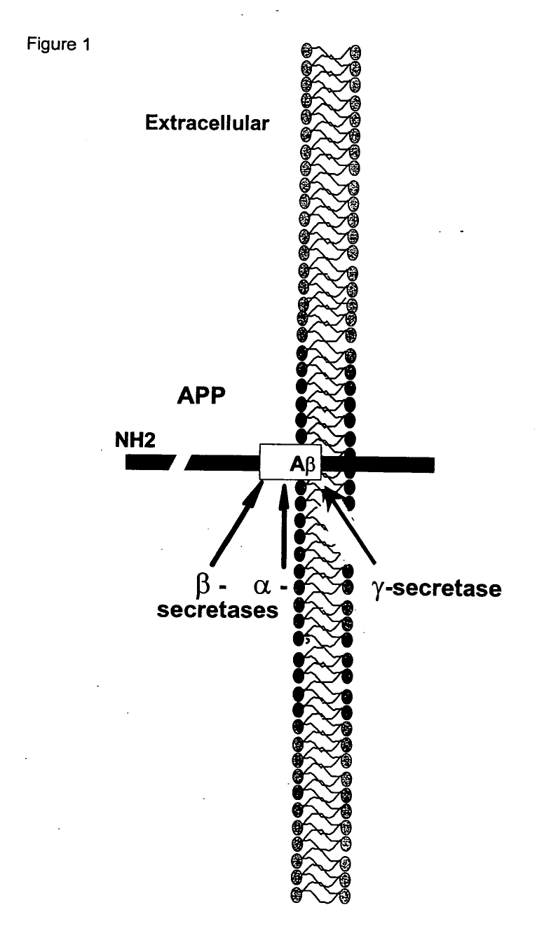

APP is an integral transmembrane protein, which firstly traverses the lipid, bilayer. By far the largest part of the protein is extracellular, while the shorter C-terminal domain is directed into the cytosol (FIG. 1). APP is the substrate of three different proteases: α-secretase, β-secretase and γ-secretase. Within APP, about two thirds of the Aβ-peptide originates from the extracellular domain and about one third from the transmembrane domain.

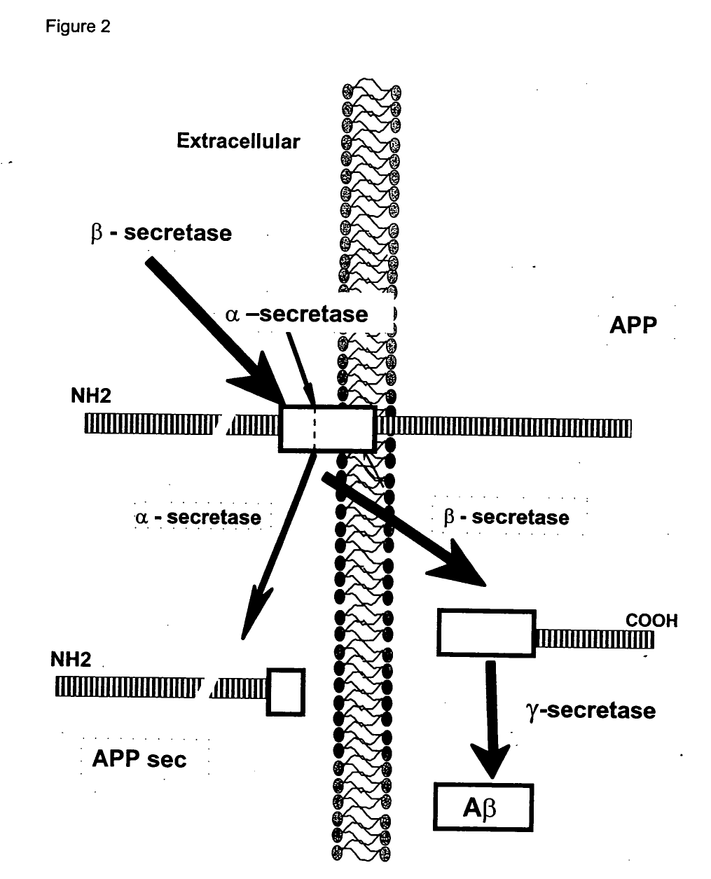

Beside the membrane-linked APP, a secreted form of APP can be detected which consists of the large ectodomain of APP and is designated as APP_{sec} (“secreted APP”). APP_{sec} is formed from APP by proteolytic cleavage, which is effected by the α-secretase (FIG. 2). This proteolytic cleavage takes place within the amino acid sequence of the Aβ-peptide (after amino acid residue 16 of the Aβ-peptide). Proteolysis of APP by the α-secretase thus excludes the formation of the Aβ-peptide.

The Aβ-peptide can thus only be formed from APP in an alternative processing route. It is postulated that two proteases are involved in this processing, one protease, which is designated as β-secretase, cleaving at the N-terminus of the Aβ-peptide in the APP and the second protease, which is designated as γ-secretase, releasing the C-terminus of the Aβ-peptide (Kang, J. et al., (1987) Nature, 325, 733 (FIG. 2).

There are many indications that the Aβ-peptide is a crucial factor in the occurrence of Alzheimer's disease. Inter alia, neurotoxicity of Aβ-fibrils in cell culture is postulated (Yankner, B.A. et al., (1990) Proc Natl Acad Sci USA, 87, 9020). In patients with Down's syndrome, in which APP occurs in an additional copy, the neuropathology characteristic of Alzheimer's disease also occurs even at an age of 30 years. Here, it is assumed that the overexpression of APP follows an increased conversion into the Aβ-peptide (Rumble, B. et al., (1989), N. Engl. J. Med., 320, 1446).

Furthermore the strongest indication of the central role of the Aβ-peptide in Alzheimer's disease are the familial forms of the disease. Here, mutations are found in the APP gene around the area of the secretase cleavage sites or in two further AD-associated genes (presenilins), which in cell culture lead to a significant increase in Aβ production (Scheuner, D. et al., (1996), Nature Medicine, 2, 864).

There are a number of indications that APP is first cleaved by the β-secretase, to serve thereafter as a substrate for γ-secretase (Maruyama, K. Y. et al., (1994) Biochem. Biophys Res Commun, 202, 1517; Estus, S. et al., (1992), Science, 255, 726). The γ-secretase therefore has a crucial role in the formation of the Aβ-peptide. A demonstration of the activity of the γ-secretase which is customarily used is the detection of the Aβ-peptide, which, however, frequently turns out to be difficult.

An important reason for this is that only a small part of APP is converted into the Aβ-peptide (Simons M, et al., Neurosci (1996) 1;16(3):899-908). Moreover, the Aβ-peptide is a small breakage fragment of about 4 kDa and, on account of its hydrophobic character, has a great tendency to self-aggregation so that it easily precipitates under physiological conditions (Hilbich, C. et al., (1991) J. Mol. Biol., 218, 149).

The short, 47 amino acid-long C-terminal tail of APP is exposed to the cytosol and is the site of interaction for molecular adaptors containing PTB domains, named Fe65, X11, m-dab and Jip1. Since the binding of these proteins is dependent on the YENPTY sequence on APP, it is expected that their interactions are not simultaneous (T. Russo et al. (1998) FEBS Left. 434-1-7; N. Zambrano et al. (1997) J. Biol. Chem. 272, 6399-6405). Indeed, it has been shown, in cultured cells, that Fe65 and X11 proteins have opposite effects on the proteolytic processing of APP (Sabo S. et al. (1999) J. Biol. Chem. 274, 7952-7957; Borg J.P. et al. (1998) J. Biol. Chem. 273, 14761-14766). A possible explanation for these findings may reside in the recruitment, by either Fe65 or X11, of APP in different macromolecular complexes, depending on the interaction of different sets of proteins with the other protein-protein interaction domains of the two adaptors.

The APP cytodomain contains various serine and threonine residues, which are phosphorylated in vitro by different kinase activities. Thr668 is a main site of phosphorylation in vivo, being phosphorylated by neuronal cyclin-dependent kinases, Cdk5 in neurons (lijima, K. et al. (2000) J. Neurochem. 75, 1085-1091), cdc2 kinase in dividing cells (Suzuki, T. et al. (1994) EMBO J. 13, 1114-1122; Oishi, M. et al. (1997) Mol. Med. 3, 111-123), and glycogen synthase kinase 3b (GSK3b) and stress-activated protein kinase 1b (SAP kinase1b) in vitro (Aplin, E. E. et al. (1996) J. Neurochem. 67, 699-707; Standen, C. L. et al. (2001) J. Neurochem. 76, 316-320). It has been shown that phosphorylation of APP regulates neurite extension in differentiating PC12 cells (Ando, K. et al. (1999) J. Neurosci. 19, 4421-4427), while, at the molecular level, APP phosphorylation may regulate the binding to the PTB domain-containing adaptors and its processing (Ando K. et al. (2001) J Biol. Chem., 276, 40353-40361).

Previously it was demonstrated that APP is phosphorylated at tyrosine 682 in cells expressing an active form of the Abl non receptor tyrosine kinase (Zambrano N. et al. (2001) J Biol. Chem. 276, 19787-92); active Abl is recruited in proximity to APP by Fe65, which may bind simultaneously these two proteins through its WW domain (Abl) and PTB2 domain (APP). Phosphorylation of Tyr682 of APP generates a docking site for the SH2 domain of Abl and, in fact stable complexes between APP and Abl are formed.

In order to test the hypothesis that tyrosine phosphorylation regulates pp60c-src biological activity, Kmiecik and Shalloway (1987) Cell 49, 65-73 described the construction and study of pp60c-src mutants in which Tyr 527 and Tyr 416 were separately or coordinately altered to phenylalanine. Tyr—Phe 527 mutation strongly activated pp60c-src transforming and kinase activities, whereas the additional introduction of a Tyr—Phe 416 mutation suppressed these activities. Tyr—Phe 416 mutation of normal pp60c-src eliminated its partial transforming activity, which suggests that transient or otherwise restricted phosphorylation of Tyr 416 is important for pp60c-src function even though stable phosphorylation is not observed in vivo. Normally, pp60c-src is phosphorylated in vivo at tyrosine 527, a residue not present in pp60v-src (its transforming homolog), and not at tyrosine 416, its site of in vitro autophosphorylation.

Williamson et al. (2002) J. Neurochem. 22, 10-20) described the rapid phosphorylation of neuronal proteins including Tau and Focal adhesion kinase in response to amyloid-β peptide exposure and an involvement of Src family protein kinases.

Slack, B. E. and Berse; B. (1998) described in Society for Neuroscience Abstracts, Vol. 24, No. 1-2, pp. 208 (Conference/Meeting Information: 28^{th} Annual Meeting of the Society for Neuroscience, Part 1, Los Angeles, Calif., USA, Nov. 7-12, 1998 Society for Neuroscience, ISSN: 0190-5295) a role for tyrosine kinases in the stimulation of APP release by action of muscarinic m3 acetylcholine receptors. They described a role for Src tyrosine kinase in the regulation of APP_{sec} release by muscarinic receptors. They demonstrated that an increase in the active form of Src leads to a decrease in secAPP.

The present invention provides methods of identifying therapeutic agents for the treatment of Alzheimer's disease.

One embodiment of the invention provides methods for identifying inhibitors of Src activity, whereby the methods comprise the steps

a) providing a Src protein (i.e. a Src encoding DNA under control of an expression element) and

b) determining if a compound inhibits the activity of Src.

The Src protein could be any mammalian Src protein, preferably human Src (SEQ ID NO. 1 (isoform 1) and SEQ ID NO. 2 (isoform 2)) or rodent Src (SEQ ID NO. 3). The protein could be obtained by expressing human Src or rodent Src cDNA, by using e. g. sequences SEQ ID NO. 4 or SEQ ID NO. 5. In addition, one of the sequences deposited under Genbank Accession No. M17031 or BC011566 can be used.

For the method preferably a mammalian cell or cell line is used in which Src is expressed, either naturally or because the cell/cell line is genetically engineered. In a particular embodiment primary cultures of neurones are used.

Another embodiment of the invention relates to a method of identifying compounds, which inhibit Src expression. The method comprises the steps

a) providing a sequence which regulates Src expression (i.e. a Src promotor sequence) and

b) determining if a compound inhibits the expression of Src protein.

The sequence which regulates Src expression is preferably linked to a reporter gene or a reporter gene construct. Such reporter gene can easily be used to determine, if a compound inhibits the expression of Src. Another possibility is the use of the Src gene. The region of chromosome 20 including the Src gene is deposited under Genbank Accession no AL133293.

An house-keeping promoter, which can be used as a sequence which regulates Src expression, has been described, as well as an alternative promoter regulated by the Hepatic Nuclear Factor-1 a (Bonham et al. (2000) J. Biol. Chem. 275, 37604) (Genbank accession number AF272982).

Active compounds, this means compounds, which inhibit Src expression or which inhibit Src protein activity, can be used as pharmaceuticals. Such compounds can be used for the preparation of a pharmaceutical for the treatment or prevention of Alzheimer's disease.

Another embodiment of the invention relates to the use of compounds identified by one of the forgoing methods for preparing a pharmaceutical for the treatment of Alzheimer's disease.

The invention further relates to a method of preparing a pharmaceutical for treatment of Alzheimer's disease comprising the steps

a) identification of a therapeutic compound by the use of one of the methods described above;

b) optimisation of the identified compound; and

c) formulation of the optimised compound.

Another embodiment of the invention relates to the use of Src inhibitors known in the art for the preparation of a pharmaceutical for the treatment or the prevention of Alzheimer's disease. The invention relates to the use of PP2 (4-amino-5-(4-chlorophenyl)-7-(t-butyl)pyrazolo[3,4d]pyrimidine) as pharmaceutical. The invention further relates to the use of PP2 for the preparation of a pharmaceutical for the treatment of Alzheime'rs disease.

Another embodiment of the invention refers to the use of PP2 for the preparation of a pharmaceutical for the treatment of Alzheimer's disease.

The new results demonstrate, that APP is phosphorylated not only by Abl. In fact Src kinase is also responsible for APP tyrosine phosphorylation. It is expected that tyrosine phosphorylation of APP alters the binding properties of its cytodomain, and then the processing of APP could be consequently modified. If so, this will prove useful the use of currently available tyrosine kinase inhibitors, and to design new ones for pharmacological modulation of the release of APP processing products in cellular and animal models of Alzheimer's disease.

Taken together, the results from the following examples suggest that exogenous Src activity is responsible for activation of a pathway resulting in increased Aβ secretion. This also suggests that PP2 could be used as a pharmacological agent able to reduce Aβ secretion in cellular and animal systems resembling AD status.

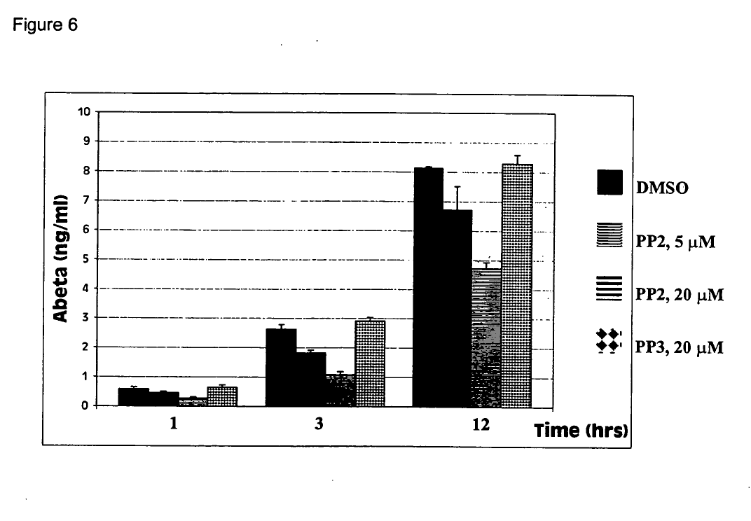

Therefore, a line of CHO cells stably overexpressing APP751 form was used, which secretes discrete levels of Aβ. If secretion involves endogenous Src activity, PP2 should be able to inhibit production of Aβ also in this cellular system. This is indeed the case since, as shown in FIG. 6, PP2 reduces in a dose-dependent manner the amount of Aβ at the three time points tested (1, 3 and 12 hours), while PP3, used at the highest concentration, has no effect.

These experiments clearly outline the relevance of Src activation in Aβ secretion and constitute the basis to try pharmacological applications for tyrosine kinase inhibitors in the treatment of AD. Screening of such inhibitors and their optimization will lead to the development of potential, innovative therapeutic agents for treatment of Alzheimer's disease.

### EXAMPLES

### Example 1

Cell Culture, Transfections and Treatments

Human embryonic kidney cells (HEK293) were cultured in Dulbecco's modified minimal medium supplemented with 10% fetal calf serum at 37° C. in a 5% CO₂ atmosphere. For transfection, cells were grown for 16 hours in antibiotic-free medium, and transfected with Lipofectamine 2000 (Invitrogen) according to manufacturer's instructions. The total amount of DNA was maintained constant by addition of empty vector DNA. 48 hours after transfection, culture medium were recovered, while cells were harvested in ice-cold phosphate buffer saline (PBS) and gently lysed in lysis buffer (50 mM Tris/HCl, pH 7.5, 150 mM NaCl, 0.5% Triton X-100, 10% glycerol, 50 mM NaF, 1mM Na vanadate), in the presence of a protease inhibitor cocktail (Complete, EDTA-free, Roche). The extracts were clarified by centrifugation at 16,000×g at 4° C., and the protein concentration determined by the Bio-Rad protein assay according to manufacturer's instructions. For metabolic labelling, 36 hours after transfection, cells were incubated for 30 minutes in medium without methionine and cysteine, then the medium was replaced with fresh medium containing 80 μCi/ml of ³⁵S-methionine/cysteine mixture (Promix, 1000 Ci/mmol, Amersham Pharmacia Biotech). Pulse was for 30 minutes, then the radioactive medium was replaced with complete medium for 90 minutes. CHO cells expressing wild-type APP751 have been described (N. Zambrano et al. (1997) J. Biol. Chem. 272, 6399-6405). Immuno-precipitations were performed either on culture medium, or on cellular lysates with 1 μg/sample of the 6E10 monoclonal antibody (Signet). Radioactive samples were resolved on 10% SDS-PAGE and quantitated with a Storm 840 phosphorimager (Amersham Pharmacia Biotech). PP2 and PP3 (Hanke, J.H. et al. (1996) J. Biol. Chem. 271, 695-701) were purchased from Calbiochem and dissolved in DMSO; treatments were performed on transfected cells for 48 hours with vehicle alone, or with 1, 5 and 20 μM PP2 or PP3. Concentration of DMSO was the same in all samples. Treatments on CHO/APP751 cells was made with DMSO, or PP2 (5 and 20 μM), or PP3 (20 μM).

### Example 2

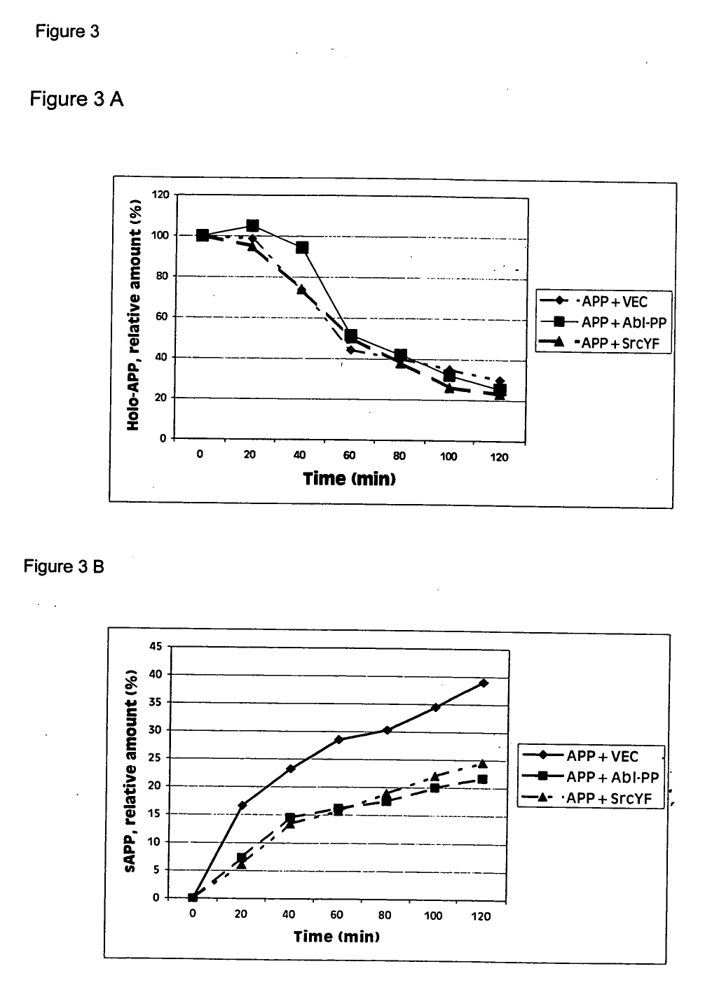

HEK293 cells were transfected with APP695 expression vectors. This cellular system was used because these cells are transfected to high efficiency, and because they express the whole set of processing activities required for APP maturation. In fact, upon transfection of human APP695 expression vectors, both α-secretase product (APP_{sec}) and β-γ-secretase products (βP) accumulate in the culture medium. HEK293 cells were co-transfected with APP and either empty vector, or active Abl (Abl-PP), or active Src (SrcYF) kinases, and the secretion of APP_{sec} and β were evaluated. Transfected cells have been pulse-labelled with ³⁵S-Met/Cys mix for 30′, and chased with cold amino acids for 90′. Both medium and protein extracts have been used in immunoprecipitation experiments for quantitative dosage of radiolabeled APP_{sec} and holo-APP, respectively. While the stability of holo-APP was not affected by co-transfection with either Abl-PP or SrcYF active kinases (FIG. 3A), a reduction in the relative amounts of APP_{sec}, normalised to the corresponding holo-APP synthesised during the pulse period was observed in both cases (FIG. 3B). This can be interpreted as a decrease of the activity of the α-secretase pathway upon co-transfection of APP with the active kinases.

### Example 3

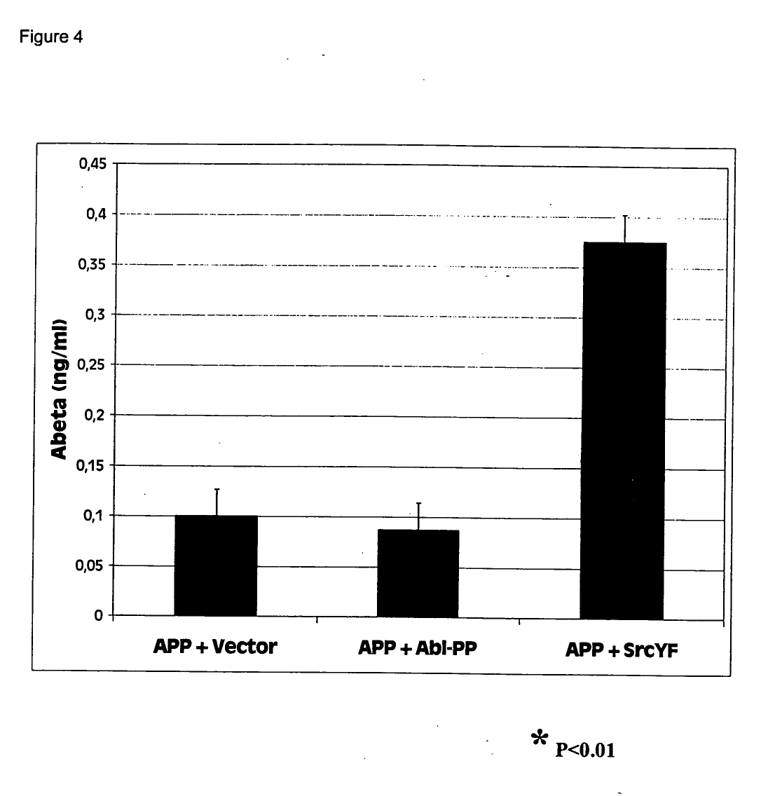

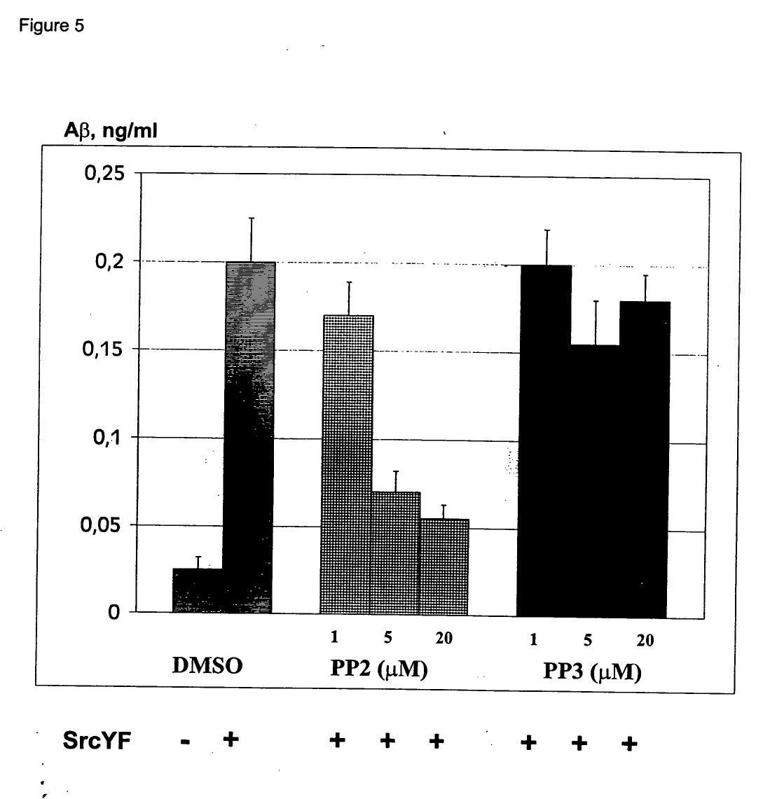

Next, the accumulation of Aβ from cells transfected as above was investigated. The dosage was performed by sandwich ELISA assays on culture medium at 48 hours after transfection (FIG. 4). The assay clearly shows that in Src-expressing cells the amount of Aβ secreted is more elevated than in the presence of the control vector, or Abl-PP. The mechanism by which active Src elicits such a dramatic increase in Aβ secretion is not clear. However, it might depend on phosphorylation of some other proteins, different from APP, since tyrosine phosphorylation of APP by Abl-PP does not result in increased Aβ levels. Accordingly, a similar behaviour is obtained with various APP mutants bearing tyrosine substitutions to either glycine or phenylalanine (data not shown). Anyway, the rise in Aβ levels does depend on the tyrosine kinase activity of Src, since the accumulation of Aβ is sensitive to treatment by the Src-family specific inhibitor PP2. In fact, as shown in FIG. 5, the exposure of transfected cells for 48 hours to increasing concentrations of PP2 results in dose-dependent decrease of secreted AP. Either PP3, an inactive analog of PP2, or vehicle alone, do not affect Aβ rise by SrcYF transfection.

Taken together, these observations suggest that exogenous Src activity is responsible for activation of a so far unidentified pathway resulting in increased Aβ secretion.

### REFERENCES

1) T. Russo et al. (1998) FEBS Left. 434,1-7.

2) N. Zambrano et al. (1997) J. Biol. Chem. 272, 6399-6405.

3) Sabo S. et al. (1999) J. Biol. Chem. 274, 7952-7957.

4) Borg J.P. et al. (1998) J. Biol. Chem. 273, 14761-14766.

5) lijima, K. et al. (2000) J. Neurochem. 75, 1085-1091.

6) Suzuki, T. et al. (1994) EMBO J. 13, 1114-1122.

7) Oishi, M. et al. (1997) Mol. Med. 3, 111-123.

8) Aplin, E. E. et al. (1996) J. Neurochem. 67, 699-707.

9) Standen, C. L. et al. (2001) J. Neurochem. 76, 316-320.

10) Ando, K. et al. (1999) J. Neurosci. 19, 4421-4427.

11) Ando K. et al. (2001) J Biol Chem.;276, 40353-40361.

12) Zambrano N. et al. (2001) J Biol Chem 276, 19787-19792.

13) Hanke, J.H. et al. (1996) J. Biol. Chem. 271, 695-701.

TABLE 1

Protein sequence of isoform 1 of human Src protein
(SEQ ID NO. 1)

MGSNKSKPKDASQRRRSLEPAENVHGAGGGAFPASQTPSKPASADGHRGPSAAFAPAAA
EPKLFGGFNSSDTVTSPQRAGPLAGGVTTFVALYDYESRTETDLSFKKGERLQIVNNTRKVD
VREGDWWLAHSLSTGQTGYIPSNYVAPSDSIQAEEWYFGKITRRESERLLLNAENPRGTFL
VRESETTKGAYCLSVSDFDNAKGLNVKHYKIRKLDSGGFYITSRTQFNSLQQLVAYYSKHAD
GLCHRLTTVCPTSKPQTQGLAKDAWEIPRESLRLEVKLGQGCFGEVWMGTWNGTTRVAIK
TLKPGTMSPEAFLQEAQVMKKLRHEKLVQLYAVVSEEPIYIVTEYMSKGSLLDFLKGETGKYL
RLPQLVDMAAQIASGMAYVERMNYVHRDLRAANILVGENLVCKVADFGLARLIEDNEYTARQ
GAKFPIKWTAPEAALYGRFTIKSDVWSFGILLTELTTKGRVPYPGMVNREVLDQVERGYRMP
CPPECPESLHDLMCQCWRKEPEERPTFEYLQAFLEDYFTSTEPQYQPGENL

TABLE 2

Protein sequence of isoform 2 of human Src
(SEQ ID NO. 2)

MGSNKSKPKDASQRRRSLEPAENVHGAGGGAFPASQTPSKPASADGHRGPSAAFAPAAA
EPKLFGGFNSSDTVTSPQRAGPLAGGVTTFVALYDYESRTETDLSFKKGERLQIVNNTEGD
WWLAHSLSTGQTGYIPSNYVAPSDSIQAEEWYFGKITRRESERLLLNAENPRGTFLVRESET
TKGAYCLSVSDFDNAKGLNVKHYKIRKLDSGGFYITSRTQFNSLQQLVAYYSKHADGLCHRL
TTVCPTSKPQTQGLAKDAWEIPRESLRLEVKLGQGCFGEVWMGTWNGTTRVAIKTLKPGT
MSPEAFLQEAQVMKKLRHEKLVQLYAVVSEEPIYIVTEYMSKGSLLDFLKGETGKYLRLPQL
VDMAAQIASGMAYVERMNYVHRDLRAANILVGENLVCKVADFGLARLIEDNEYTARQGAKF
PIKWTAPEAALYGRFTIKSDVWSFGILLTELTTKGRVPYPGMVNREVLDQVERGYRMPCPPE
CPESLHDLMCQCWRKEPEERPTFEYLQAFLEDYFTSTEPQYQPGENL

TABLE 3

Sequence of mouse Src protein
(SEQ ID NO. 3)

MGSNKSKPKDASQRRRSLEPSENVHGAGGAFPASQTPSKPASADGHRGPSAAFVPPAAEP
KLFGGFNSSDTVTSPQRAGALAGGVTTFVALYDYESRTETDLSFKKGERLQIVNNTRKDVR
EGDWWLAHSLSTGQTGYIPSNYVAPSDSIQAEEWYFGKITRRESERLLLNAENPRGTFLVR
ESETTKGAYCLSVSDFDNAKGLNVKHYKIRKLDSGGFYITSRTQFNSLQQLVAYYSKHADGL
CHRLTTVCPTSKPQTQGLAKDAWEIPRESLRLEVKLGQGCFGEVWMGTWNGTTRVAIKTLK
PGTMSPEAFLQEAQVMKKLRHEKLVQLYAVVSEEPIYIVTEYMNKGSLLDFLKGETGKYLRL
PQLVDMSAQIASGMAYVERMNYVHRDLRAANILVGENLVCKVADFGLARLIEDNEYTARQG
AKFPIKWTAPEAALYGRFTIKSDVWSFGILLTELTTKGRVPYPGMVNREVLDQVERGYRMPC
PPECPESLHDLMCQCWRKEPEERPTFEYLQAFLEDYFTSTEPQYQPGENL

TABLE 4

cDNA sequence encoding human Scr protein
(SEQ ID NO. 4)

| | |
|---|---|
| 1 | catcgaggtt ttgagaggct aactctccca aaaaggacca tgggtagcaa caagagcaag |
| 61 | cccaaggatg ccagccagcg gcgccgcagc ctggagcccg ccgagaacgt gcacggcgct |
| 121 | ggcgggggcg ctttccccgc ctcgcagacc cccagcaagc cagcctcggc cgacggccac |
| 181 | cgcggcccca gcgcggcctt cgcccccgcg gccgccgagc ccaagctgtt cggaggcttc |
| 241 | aactcctcgg acaccgtcac ctccccgcag agggcgggcc cgctggccgg tggagtgacc |
| 301 | acctttgtgg ccctctatga ctatgagtct aggacggaga cagacctgtc cttcaagaaa |
| 361 | ggcgagcggc tccagattgt caacaacaca gagggagact ggtggctggc ccactcgctc |
| 421 | agcacaggac agacaggcta catccccagc aactacgtgg cgccctccga ctccatccag |
| 481 | gctgaggagt ggtattttgg caagatcacc agacgggagt cagagcggtt actgctcaat |
| 541 | gcagagaacc cgagagggac cttcctcgtg cgagaaagtg agaccacgaa aggtgcctac |
| 601 | tgcctctcag tgtctgactt cgacaacgcc aagggcctca acgtgaagca ctacaagatc |
| 661 | cgcaagctgg acagcggcgg cttctacatc acctcccgca cccagttcaa cagcctgcag |
| 721 | cagctggtgg cctactactc caaacacgcc gatggcctgt gccaccgcct caccaccgtg |
| 781 | tgccccacgt ccaagccgca gactcagggc ctggccaagg atgcctggga gatccctcgg |
| 841 | gagtcgctgc ggctggaggt caagctgggc cagggctgct ttggcgaggt gtggatgggg |
| 901 | acctggaacg gtaccaccag ggtggccatc aaaaccctga agcctggcac gatgtctcca |
| 961 | gaggccttcc tgcaggaggc ccaggtcatg aagaagctga ggcatgagaa gctggtgcag |
| 1021 | ttgtatgctg tggtttcaga ggagcccatt tacatcgtca cggagtacat gagcaagggg |
| 1081 | agtttgctgg actttctcaa gggggagaca ggcaagtacc tgcggctgcc tcagctggtg |
| 1141 | gacatggctg ctcagatcgc ctcaggcatg gcgtacgtgg agcggatgaa ctacgtccac |
| 1201 | cgggaccttc gtgcagccaa catcctggtg ggagagaacc tggtgtgcaa agtggccgac |
| 1261 | tttgggctgg ctcggctcat tgaagacaat gagtacacgg cgcggcaagg tgccaaattc |
| 1321 | cccatcaagt ggacggctcc agaagctgcc ctctatggcc gcttcaccat caagtcggac |
| 1381 | gtgtggtcct tcgggatcct gctgactgag ctcaccacaa agggacgggt gccctaccct |
| 1441 | gggatggtga accgcgaggt gctggaccag gtggagcggg gctaccggat gccctgcccg |
| 1501 | ccggagtgtc ccgagtccct gcacgacctc atgtgccagt gctggcggaa ggagcctgag |
| 1561 | gagcggccca ccttcgagta cctgcaggcc ttcctggagg actacttcac gtccaccgag |
| 1621 | ccccagtacc agcccgggga gaacctctag gcacaggcgg gcccagaccg gcttctcggc |
| 1681 | ttggatcctg ggctgggtgg cccctgtctc ggggcttgcc ccactctgcc tgcctgctgt |
| 1741 | tggtcctctc tctgtggggc tgaattgcca ggggcgaggc ccttcctctt tggtggcatg |
| 1801 | gaaggggctt ctggacctag ggtggcctga gagggcggtg ggtatgcgag accagcacgg |
| 1861 | tgactctgtc cagctcccgc tgtggccgca cgcctctccc tgcactccct cctggagctc |
| 1921 | tgtgggtctc tggaagagga accaggagaa gggctggggc cggggctgag ggtgcccttt |
| 1981 | tccagcctca gcctactccg ctcactgaac tccttcccca cttctgtgcc acccccggtc |
| 2041 | tatgtcgaga gctggccaaa gagcctttcc aaagaggagc gatgggcccc tggccccgcc |
| 2101 | tgcctgccac cctgcccctt gccatccatt ctggaaacac ctgtaggcag aggctgccga |
| 2161 | gacagaccct ctgccgctgc ttccaggctg ggcagcacaa ggccttgcct ggcctgatga |
| 2221 | tggtgggtgg gtgggatgag taccccctca aaccctgccc tccttagacc tgagggaccc |
| 2281 | ttcgagatca tcacttcctt gcccccattt cacccatggg gagacagttg agagcgggga |
| 2341 | tgtgacatgc ccaaggccac ggagcagttc agagtggagg cgggcttgga acccggtgct |
| 2401 | ccctctgtca tcctcaggaa ccaacaattc gtcggaggca tcatggaaag actgggacag |
| 2461 | cccaggaaac aaggggtctg aggatgcatt cgagatggca gattcccact gccgctgccc |
| 2521 | gctcagccca gctgttggga acagcatgga ggcagatgtg gggctgagct ggggaatcag |
| 2581 | ggtaaaaggt gcaggtgtgg agagagaggc ttcaatcggc ttgtgggtga tgtttgacct |
| 2641 | tcagagccag ccggctatga aagggagcga gcccctcggc tctggaggca atcaagcaga |
| 2701 | catagaagag ccaagagtcc aggaggccct ggtcctggcc tccttccccg tactttgtcc |
| 2761 | cgtggcattt caattcctgg ccctgttctc ctccccaagt cggcaccctt taactcatga |
| 2821 | ggagggaaaa gagtgcctaa gcgggggtga aagaggacgt gttacccact gccatgcacc |
| 2881 | aggactggct gtgtaacctt gggtggcccc tgctgtctct ctgggctgca gagtctgccc |
| 2941 | cacatgtggc catggcctct gcaactgctc agctctggtc caggccctgt ggcaggacac |
| 3001 | acatggtgag cctagccctg ggacatcagg agactgggct ctggctctgt tcggcctttg |
| 3061 | ggtgtgtggt ggattctccc tgggcctcag tgtgcccatc tgtaaagggg cagctgacag |
| 3121 | tttgtggcat cttgccaagg gtccctgtgt gtgtgtatgt gtgtgcatgt gtgcgtgtct |
| 3181 | ccatgtgcgt ccatatttaa catgtaaaaa tgtccccccc gctccgtccc ccaaacatgt |
| 3241 | tgtacatttc accatggccc cctcatcata gcaataacat tcccactgcc aggggttctt |
| 3301 | gagccagcca ggccctgcca gtggggaagg aggccaagca gtgcctgcct atgaaatttc |
| 3361 | aacttttcct ttcatacgtc tttattaccc aagtcttctc ccgtccattc cagtcaaatc |
| 3421 | tgggctcact caccccagcg agctctcaaa tccctctcca actgcctaag gccctttgtg |
| 3481 | taaggtgtct taatactgtc cttttttttt ttttaacagt gttttgtaga tttcagatga |
| 3541 | ctatgcagag gcctggggga cccctggctc tgggccgggc ctggggctcc gaaattccaa |
| 3601 | ggcccagact tgcggggggt gggggggtat ccagaattgg ttgtaaatac tttgcatatt |
| 3661 | gtctgattaa acacaaacag acctcagaaa aaaaaaaaaa aaaaaaaaaa a |

TABLE 5
Mouse Src cDNA sequence (SEQ ID NO. 5)

| | |
|---|---|
| 1 | atgggcagca acaagagcaa gcccaaggac gccagccagc ggcgccgcag cctggagccc |
| 61 | tcggaaaacg tgcacggggc agggggcgcc ttcccggcct cacagacacc gagcaagccc |
| 121 | gcctccgccg acggccaccg cgggcccagc gccgccttcg tgccgcccgc ggccgagccc |
| 181 | aagctcttcg gaggcttcaa ctcctcggac accgtcacct ccccgcagag ggcgggcgct |
| 241 | ctggcaggtg gggtgaccac ctttgtggcc ctctatgact atgagtcacg gacagagact |
| 301 | gacctgtcct tcaagaaagg ggagcggctg cagattgtca ataacacgag gaaggtggat |
| 361 | gtcagagagg gagactggtg gctggcacac tcgctgagca cgggacagac cggttacatc |
| 421 | cccagcaact atgtggcgcc ctccgactcc atccaggctg aggagtggta ctttggcaag |
| 481 | atcactagac gggaatcaga gcggctgctg ctcaacgccg agaacccgag agggaccttc |
| 541 | ctcgtgaggg agagtgagac cacaaaaggt gcctactgcc tctctgtatc cgacttcgac |
| 601 | aatgccaagg gcctaaatgt gaaacactac aagatccgca agctggacag cggcggtttc |
| 661 | tacatcacct cccgcaccca gttcaacagc ctgcagcagc tcgtggctta ctactccaaa |
| 721 | catgctgatg gcctgtgtca ccgcctcact accgtatgtc ccacatccaa gcctcagacc |
| 781 | cagggattgg ccaaggatgc gtgggagatc ccccgggagt ccctgcggct ggaggtcaag |
| 841 | ctgggccagg gttgcttcgg agaggtgtgg atggggacct ggaacggcac cacgagggtt |
| 901 | gccatcaaaa ctctgaagcc aggcaccatg tccccagagg ccttcctgca ggaggcccaa |
| 961 | gtcatgaaga aactgaggca cgagaaactg gtgcagctgt atgctgtggt gtcggaagaa |
| 1021 | cccatttaca ttgtgacaga gtacatgaac aaggggagtc tgctggactt tctcaagggg |
| 1081 | gaaacgggca aatatttgcg gctaccccag ctggtggaca tgtctgctca gatcgcttca |
| 1141 | ggcatggcct atgtggagcg gatgaactat gtgcaccggg accttcgagc cgccaatatc |
| 1201 | ctagtagggg agaacctggt gtgcaaagtg gccgactttg ggttggcccg gctcatagaa |
| 1261 | gacaacgaat acacagcccg gcaaggtgcc aaattcccca tcaagtggac cgcccctgaa |
| 1321 | gctgctctgt acggcaggtt caccatcaag tcggatgtgt ggtcctttgg gattctgctg |
| 1381 | accgagctca ccactaaggg aagagtgccc tatcctggga tggtgaaccg tgaggttctg |
| 1441 | gaccaggtgg agcggggcta ccggatgcct tgtccccccg agtgccccga gtccctgcat |
| 1501 | gaccttatgt gccagtgctg gcggaaggag cccgaggagc ggcccacctt cgagtacctg |
| 1561 | caggccttcc tggaagacta ctttacgtcc actgagccac agtaccagcc cggggagaac |
| 1621 | ctatag |

what is claimed is:

1. A method for identifying a therapeutic compound for the treatment of Alzheimer's disease comprising the step of identifying a Src protein inhibitor.

2. The method of claim 1 wherein said identifying step comprises the steps of:
a) providing a reporter gene operatively linked to a regulatory nucleotide sequence, wherein said regulatory nucleotide sequence regulates Src protein expression;
b) introducing a candidate compound; and
c) measuring the activity of said reporter gene in response to said candidate compound.

3. The method of claim 2 wherein said reporter gene is Src.

4. The method of claim 2, wherein said regulatory nucleotide sequence is the Src promoter.

5. The method of claim 2 wherein said regulatory nucleotide sequence regulates a Src protein selected from the group consisting of: human Src and mouse Src.

6. The method of claim 1 wherein said Src protein is selected from the group consisting of: human Src and mouse Src.

7. The method of claim 1, wherein said Src protein comprises an amino acid sequence selected from the group consisting of: SEQ ID NO:1, SEQ ID NO:2, and SEQ ID NO:3.

8. The method of claim 1, comprising the steps of:
a) providing at least one mammalian cell expressing said Src protein;
b) introducing a candidate compound to said mammalian cell; and
c) measuring the activity of said Src protein in response to said candidate compound.

9 The method of claim 8, wherein said providing step (a) comprises providing a primary cell culture of neurons.

10 A therapeutic compound identified by the method of claim 1.

11 A pharmaceutical composition comprising an effective amount of the therapeutic compound of claim 10 and a pharmaceutically acceptable excipient.

12 A method for treating Alzheimer's disease comprising the step of administering the pharmaceutical composition of claim 11.

13. A pharmaceutical composition comprising an effective amount of PP2 and a pharmaceutically acceptable excipient.

14. A method for treating Alzheimer's disease comprising the step of administering the pharmaceutical composition of claim 13.
